(19) United States
(12) Patent Application Publication
(10) Pub. No.: US 2026/0215764 A1
(43) Pub. Date: Jul. 30, 2026

(54) PORTABLE ULTRASOUND SYSTEM FOR IMAGE TRIGGERING AND BATTERY CONSUMPTION REDUCTION

(71) Applicant: Medtronic, Inc., Minneapolis, MN (US)

(72) Inventors: Julio Jenaro Rodriguez, Munich (DE); Samiksha Tiwari, Brooklyn Park, MN (US)

(21) Appl. No.: 19/150,400

(22) PCT Filed: Jan. 23, 2024
(86) PCT No.: PCT/IB2024/050634

§ 371 Date: Jul. 23, 2025

Related U.S. Application Data
(60) Provisional application No. 63/482,139, filed on Jan. 30, 2023.

(51) Int. Cl. A61B 8/00; A61B 8/06

(52) U.S. Cl. CPC A61B 8/543; A61B 8/065; A61B 8/4236; A61B 8/4427; A61B 8/565

(57) ABSTRACT

An example cardiac monitor includes a low-power cardiac sensor and an ultrasound array. A processor is configured to perform feature extraction of the data from the low-power cardiac sensor to detect an onset of an arrhythmia condition. When the arrhythmia condition is detected, the processor triggers an ultrasound measurement using the ultrasound array based on detection of the onset of the arrhythmia condition.

This application claims the benefit of U.S. Provisional Patent Application Ser. No. 63/482,139, filed Jan. 30, 2023, the entire content of which is incorporated herein by reference.

### FIELD

The disclosure relates generally to medical devices and, more particularly, portable cardiac monitor devices.

### BACKGROUND

Medical devices may be used to monitor various physiological parameters of a patient. For example, some medical devices are configured to sense cardiac electrogram (EGM) signals indicative of the electrical activity of the heart via electrodes. Some medical devices are configured with photoplethysmogram (PPG) sensors for measuring blood flow parameters such as the heart rate, blood pressure, and cardiac cycle. Still other devices are configured with an ultrasound sensor array for imaging the heart, and/or for detecting physiological parameters of the heart such as structure, stroke volume, left ventricular ejection fraction, and other measurements.

### SUMMARY

In various aspects, the present disclosure provides a portable cardiac monitor device that combines an energy efficient cardiac sensor such as an EGM or PPG sensor, with an ultrasound sensor array that provides complementary data for advanced diagnosis and treatment. With energy-efficient cardiac sensing moderating the use of the more energy-intensive ultrasound array an effective diagnostic tool for various heart conditions is provided. In some examples, the energy efficient cardiac sensor is included in a patch, or other wearable or implantable device along with the ultrasound array, providing an effective and portable cardiac monitor device with a relatively long battery life and with advanced ultrasound diagnostic capabilities. One or more wearable or implantable medical devices configured according to the techniques of this disclosure, may advantageously provide continuous (e.g., periodic and/or triggered without user intervention) cardiac monitoring using both an energy efficient sensor and ultrasound for a period of at least several days and as long as several years that represents an improvement over manual monitoring by a clinician or the patient.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

Various embodiments described herein relate to an apparatus including one or more of the following: a processor; a cardiac sensor; and an ultrasound sensor, wherein the processor is configured to: detect an onset of an arrhythmia condition using the cardiac sensor; and trigger an ultrasound measurement using the ultrasound sensor based on detection of the onset of the arrhythmia condition using the cardiac sensor.

Various embodiments are described wherein the cardiac sensor comprises one of a cardiac electrogram (EGM) sensor or a photoplethysmogram (PPG) sensor.

Various embodiments are described wherein the ultrasound measurement comprises one of a blood flow measurement or a cardiac image.

Various embodiments are described wherein the apparatus comprises a wearable device or a patch.

Various embodiments are described wherein the arrhythmia condition comprises atrial fibrillation.

Various embodiment additionally include a battery, wherein conducting the ultrasound measurement increases power consumption on the battery, and wherein, subsequent to conducting the ultrasound measurement, the processor is configured to cease using the ultrasound sensor, whereby the power consumption on the battery is decreased.

Various embodiments described herein relate to a medical device system including one or more of the following: an implantable medical device including: a plurality of electrodes to configured detect a cardiac electrical signal of a patient, communication circuitry configured to communicate with at least one other device; and processing circuitry configured to: identify a cardiac episode in the cardiac electrical signal of the patient, and transmit an instruction for the external device to begin monitoring the cardiac episode.

Various embodiments are described wherein the implantable medical device comprises the processing circuitry.

Various embodiments are described wherein, in transmitting the instruction, the processing circuitry is configured to transmit the instruction via the communication circuitry directly to the external device.

Various embodiments are described wherein the cardiac episode is an arrythmia episode.

Various embodiments are described wherein the arrythmia episode is an atrial fibrillation episode.

Various embodiments are described wherein the external device comprises an ultrasound sensor.

Various embodiments described herein relate to a sensor patch comprising: a sensor device; a power supply; and a processor configured to: receive an indication that a cardiac sensor has detected a cardiac event in a patient, utilize the sensor device to produce a measurement of the cardiac event, whereby a power draw on the power supply is increased, and cease utilization of the sensor device after the measurement of the cardiac event is produced, whereby a power draw on the power supply is decreased.

Various embodiment additionally include input/output circuitry, wherein the cardiac sensor is external to the sensor patch and the indication is received via the input/output circuitry.

Various embodiments are described wherein the sensor device comprises an ultrasound array.

### BRIEF DESCRIPTION OF THE DRAWINGS

FIG. 1 illustrates the environment of an example medical system in conjunction with a patient in accordance with some examples of the current disclosure.

FIG. 2 is a functional block diagram illustrating an example configuration of the cardiac sensor device of the medical system of FIG. 1 in accordance with some examples of the current disclosure.

FIG. 3 is a block diagram illustrating an example system that includes a network and an external device such as a server, which may be coupled to the cardiac sensor device of FIGS. 1-2 in accordance with some examples of the current disclosure.

FIG. 4 is a flow chart illustrating an example process for triggering an ultrasound measurement based on detection of an arrhythmia condition according to some examples of the current disclosure.

Like reference characters denote like elements throughout the description and figures.

### DETAILED DESCRIPTION

Some conditions may be better detected when analyzing data obtained from multiple different sensing modalities. For example, atrial fibrillation (Afib) may be detected using a combination of electrocardiogram (ECG) data (as may be obtained from a 12-lead holter monitor; a REVEAL LINQ™ or LINQ II™ insertable cardiac monitor (ICM) available from Medtronic, Inc.; or other insertable cardiac device (ICD)) and measures such as stroke volume, left ventricular ejection fraction, and/or heart structure information (as may be obtained from an ultrasound modality, such as an ultrasound patch applied to a patient's chest). Some such modalities, such as an ultrasound patch, may have a limited power source (e.g., a battery) that would be drained by continuous application of sensing modalities (e.g., continuous or pulsed wave Doppler). With such excessive power drain, the patch may be rendered infeasible for long term and continuous monitoring of a condition such as Afib. Other combinations of sensors and monitored conditions with similar power constraints will be apparent and will benefit from the teachings presented herein.

According to various embodiments, data from first sensor that is able to operate more frequently than a second sensor is used to trigger operation of the second sensor. For example, where the first sensor detects an event (or a sufficiently high likelihood of an event) to be monitored, the second sensor is activated to capture data about the event and then deactivated to conserve power until the next such occasion for monitoring. According to some embodiments, the first sensor is an ECG sensor that detects an Afib or other cardiac event, while the second sensor is an ultrasound sensor that is activated in response to the cardiac event to gather data such as stroke volume, left ventricular ejection fraction, and/or heart structure information. In some embodiments, the two sensors may be integrated into the same device (e.g., a single patch, provided some method of communication between the two device) or may be separate devices (e.g., an ICD in communication with an ultrasound patch via a wired or wireless communication protocol, such as according to the Bluetooth low energy standard).

According to the foregoing, various embodiments present a technical improvement to the fields of cardiac and other medical monitoring as well as battery management. By utilizing one sensor to drive the operation of another, more power-intensive sensor, power constraints can be overcome to provide effectively continuous monitoring of patient events by a sensor that is not truly capable of continuous monitoring for a length of time that is feasible for the purposes of the prescribed patient monitoring. Various other technical benefits will be apparent in view of the following description.

In operation, an EGM/PPG sensor for cardiac sensing and an ultrasound sensor array for ultrasound measuring or imaging may each be placed on the same or different parts of the body of the patient. For example, the EGM/PPG cardiac sensor and the ultrasound sensor array may be physically separate from each other and may be separately placed on the patient. For example, the EGM/PPG sensor may be of the type described in, e.g., PCT application number PCT/IB2023/057429, filed Jul. 20, 2023, the entire disclosure of which is hereby incorporated herein by reference. As another example, the EGM/PPG cardiac sensor and the ultrasound sensor array may be in some cases supported by a single housing. Although primarily described in the context of an example in which a single wearable device, e.g., patch, includes both the EGM/PPG sensor and ultrasound array, one or more of the sensors may be included in one or more implantable devices in some examples.

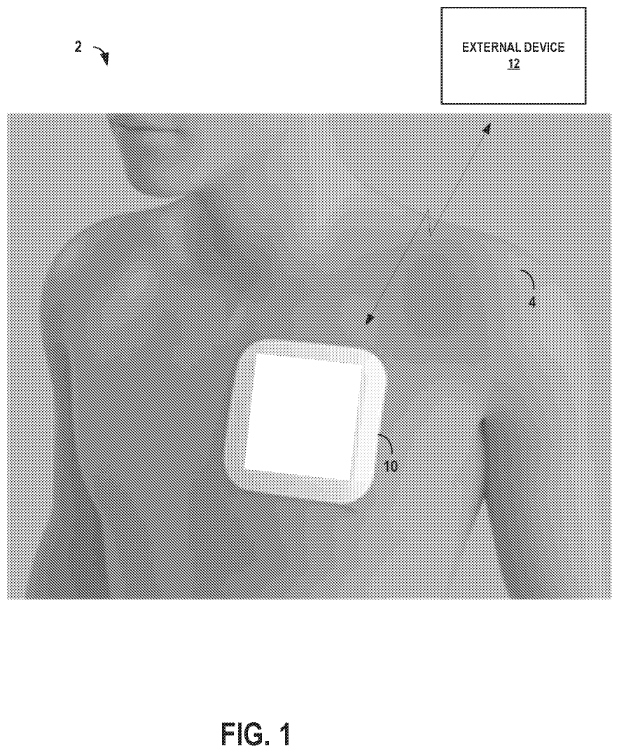

FIG. 1 illustrates the environment of an example medical system 2 in conjunction with a patient 4, in accordance with one or more techniques of this disclosure. The example techniques may be used with a cardiac monitor 10, which may be in wired or wireless communication with external device 12 and/or other devices not pictured in FIG. 1. In some examples, a cardiac monitor 10 may take the form of a patch, with a suitable adhesive to facilitate the patch to adhere to the chest of the patient 4. As described further below, cardiac monitor 10 may be a portable, battery-powered unit that lies substantially flat on the chest of the patient 4. This can facilitate the patient's wearing of the device underneath clothing during everyday activity such that real world diagnostic data may be gathered. However, cardiac monitor 10 may take other forms, and the patch form illustrated in FIG. 1 is not intended to be limiting.

External device 12 may be a computing device with a display viewable by the user and an interface for providing input to external device 12 (e.g., a user input mechanism). In some examples, external device 12 may be a notebook computer, tablet computer, workstation, one or more servers, cellular phone, smartphone, personal digital assistant, or another computing device that may run an application that enables the computing device to interact with the cardiac monitor 10.

External device 12 may be used to configure operational parameters for cardiac monitor 10. External device 12 may be used to retrieve data from cardiac monitor 10. The retrieved data may include values of physiological parameters measured by cardiac monitor 10, indications of episodes of arrhythmia or other maladies detected by cardiac monitor 10, and physiological signals recorded by cardiac monitor 10. For example, external device 12 may retrieve information related to detection of an AF episode, and in some examples may take various actions based on the AF episode such as sending an alert to a clinician.

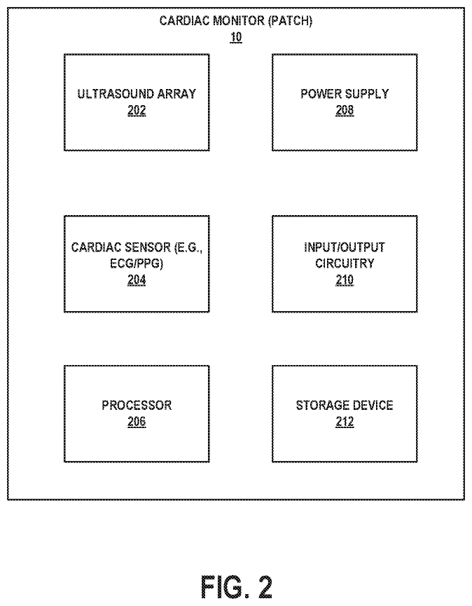

FIG. 2 is a functional block diagram illustrating an example configuration of cardiac monitor 10 in accordance with one or more techniques described herein. In the illustrated example, cardiac monitor 10 includes ultrasound array 202, cardiac sensor 204, processor 206, power supply 208, input/output circuitry 210, and storage device 212.

Ultrasound array 202 may be any suitable sensor capable of generating ultrasound and measuring one or more parameters of the patient's heart.

Ultrasound patch technology allows for measurement of parameters like stroke volume or left ventricular ejection fraction that are not possible to measure with an EGM or PPG sensor. The ultrasound array 202 can further provide heart structure for better understanding of the physical problem in the heart.

In order to obtain the heart rate information that an ECG could provide, Continuous Wave Doppler or Pulsed Wave Doppler may be used to detect the time interval between R-wave events by detecting the corresponding cardiac contractions. In these cases, the energy consumption is higher than that required by a cardiac sensor such as an EGM or PPG sensor. Thus, according to some aspects of this disclosure, a processor may determine to enable the ultrasound sensor from time to time based on measurements from a more efficient cardiac sensor that may be integrated into the cardiac monitor device.

While atrial fibrillation (AF) detection using an EGM sensor based on electrical activity of the heart is possible, such EGM measurement information alone does not provide volume or structure information. On the other hand, portable or patch ultrasounds can extend the information provided by such an EGM sensor for a more accurate diagnosis and detection. However, the energy consumption for an ultrasound measurement is higher, limiting the battery duration and therefore the continuous monitoring procedure time.

In various aspects, cardiac monitor 10 may include a cardiac sensor such as an EGM sensor or a PPG sensor. In some examples, as described further below, this cardiac sensor may be an energy-efficient tool such that continuous, periodic, or intermittent operation may occur over an extended period of time before draining the battery. For example, the cardiac sensor may be configured to operate for a period of several days or weeks before depletion of the battery.

A variety of types of medical devices sense cardiac EGMs. In some examples, EGMs may also include electrocardiograms (ECGs or EKGs). Some medical devices that sense cardiac EGMs are non-invasive, e.g., using a plurality of electrodes placed in contact with external portions of the patient, such as at various locations on the skin of the patient. The electrodes used to monitor the cardiac EGM in these non-invasive processes may be attached to the patient using an adhesive, strap, belt, or vest, as examples, and electrically coupled to a monitoring device, such as an electrocardiograph, Holter monitor, or other electronic device. The electrodes are configured to sense electrical signals associated with the electrical activity of the heart or other cardiac tissue of the patient, and to provide these sensed electrical signals to the electronic device for further processing and/or display of the electrical signals. The non-invasive devices and methods may be utilized on a temporary basis, for example to monitor a patient during a clinical visit, such as during a doctor's appointment, or for example for a predetermined period of time, for example for one day (twenty-four hours), or for a period of several days.

External devices that may be used non-invasively to sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities, and may periodically transmit collected data to a network service, such as the Medtronic Carelink™ Network.

A variety of types of medical devices sense heart rhythms using PPG. Some medical devices that use PPGs illuminate the skin at a suitable location and measure changes in light absorption via a suitable photodetector. PPG is a non-invasive diagnostic technique for monitoring physiological parameters relating to blood flow, such as a heart rhythm, blood pressure, pulse oximetry, etc.

Input/output circuitry 210 may include any suitable hardware, firmware, software, or any combination thereof for communicating with another device, such as external device 12, another networked computing device, etc. Under the control of processor 206, input/output circuitry 210 may receive downlink telemetry from, as well as send uplink telemetry to external device 12 or another device with the aid of an internal or external antenna. In addition, processor 206 may communicate with a networked computing device via an external device (e.g., external device 12) and a computer network, such as the Medtronic CareLink® Network. Input/output circuitry 210 may be wireless circuitry configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

Processor 206 may include fixed function circuitry and/or programmable processing circuitry. Processor 206 may include any one or more of a microprocessor, a controller, a digital signal processor (DSP), an application specific integrated circuit (ASIC), a field-programmable gate array (FPGA), graphics processing units (GPUs), or equivalent discrete or analog logic circuitry. In some examples, processor 206 may include multiple components, such as any combination of one or more microprocessors, one or more controllers, one or more DSPs, one or more ASICs, or one or more FPGAs, as well as other discrete or integrated logic circuitry. The functions attributed to processor 206 herein may be embodied as software, firmware, hardware or any combination thereof.

In some examples, storage device 212 includes computer-readable instructions that, when executed by processor 206, cause cardiac monitor 10 and processor 206 to perform various functions attributed to cardiac monitor 10 and processor 206 herein. Storage device 212 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 212 may store, as examples, programmed values for one or more operational parameters of cardiac monitor 10 and/or data collected by cardiac monitor 10 for transmission to another device using input/output circuitry 210. Data stored by storage device 212 and transmitted by input/output circuitry 210 to one or more other devices may include digitized cardiac EGMs, digitized PPG signals, heart rhythm classifications, ultrasound measurements, or ultrasound images, as some examples.

Processor 206 may be configured to analyze a signal from the cardiac sensor (e.g., an EGM signal or a PPG signal) and determine a heart rhythm condition of the patient. That is, the processor 206 may continuously or periodically employ a low-power cardiac sensor 204 to monitor a patient's heart, and based on the cardiac sensor 204 measurements, the processor 206 may make either pulsed ultrasound images or other ultrasound measurements triggered by events.

In some examples, processor 206 may be configured to execute instructions to analyze data from one or more cardiac sensors 204. For example, processor 206 may be configured to analyze the detected heart rhythm from a cardiac sensor (e.g., an EGM or PPG sensor) and determine a heart rhythm condition. A heart rhythm condition may be, for example, a sinus rhythm (SR), atrial fibrillation (AF), or other heart rhythm conditions. According to one example, processor 206 may store data in storage device 212 relating to the onset and duration of AF episodes.

In various aspects of this disclosure, processor 206 may make a heart rhythm condition classification as either AF or SR. That is, in coordination with the cardiac sensors 204 of the cardiac monitor 10, processor 206 may detect AF episodes as they occur. Techniques for detecting AF are known to those of ordinary skill in the art, and may include criteria related to rate and regularity of R-waves in the cardiac ECG signal. In one example, an AF detection algorithm may be based on an R-R interval pattern-based algorithm and a P-wave evidence score, which reduces false positive AF detections and leverages the evidence of a single P-wave between two R waves using morphologic processing of the ECG signal. In a further example, an AF detection algorithm may make a heart rhythm condition classification every 2 minutes, or at another suitable interval.

In some examples, processor 206 may be further configured to execute instructions to trigger an action based on the determined heart rhythm condition and/or based on an ultrasound measurement.

For example, when the processor 206 detects an arrhythmia condition (e.g., AF) using the cardiac sensor 204 and/or detects an alert condition using the ultrasound array 202, the processor 206 may send an alert to a clinician or to the patient to notify them of the arrhythmia condition.

When the processor 206 detects an arrhythmia condition (e.g., AF) using the cardiac sensor 204, the processor 206 may trigger an ultrasound measurement (e.g., any suitable measurement or imaging) using the ultrasound array 202.

Reducing the need to schedule an appointment for an ultrasound echo-cardiograph, as it can be provided in real-time.

Processor 206 may store, process, and/or output sensor data or other information at any suitable time. For example, processor 206 may be configured for event-based actions, where an event triggers the processor 206 to store, process, and/or output sensor data or other information. Such events may include the detection of the onset or predicted onset of an arrhythmia episode, such as an AF episode, for example.

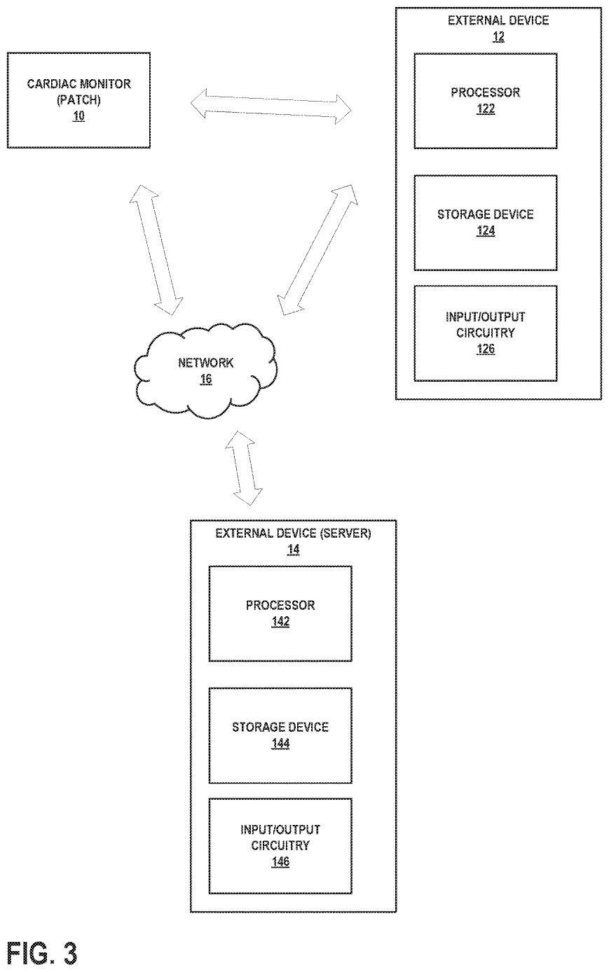

FIG. 3 is a system diagram illustrating an example system that includes a cardiac monitor (patch) 10, an external device 12, and a server 14, which may be coupled to the cardiac monitor 10 and/or the external device 12 via network 16, in accordance with one or more techniques described herein. In this example cardiac monitor 10 may use input/output circuitry 210 to communicate with external device 12 via a wired or wireless connection.

In some cases, server 14 may be configured to provide a secure storage site for data that has been collected from cardiac monitor 10 and/or external device 12. In some cases, server 14 may assemble data in web pages or other documents for viewing by trained professionals, such as clinicians, via any suitable computing device. One or more aspects of the illustrated system of FIG. 3 may be implemented with general network technology and functionality, which may be similar to that provided by the Medtronic CareLink® network. In some examples, server 14 may communicate with cardiac monitor 10 and/or external device 12 an analysis of data, such as heart rhythm condition classification and ultrasound measurements and/or images.

In some examples, the server 14 may be or may be in communication with a tablet or other smart device located with a clinician, by which the clinician may program, receive alerts from, and or interrogate cardiac monitor 10. For example, a clinician may access data collected by cardiac monitor 10, such as when patient 4 is between clinician visits, to check on a status of a medical condition. In some examples, the clinician may enter instructions for a medical intervention for patient 4 into an application executed by server 14, such as based on a status of a patient condition determined by cardiac monitor 10, external device 12, server 14, or any combination thereof, or based on other patient data known to the clinician. Server 14 may then transmit the instructions for medical intervention to the external device 12 located with the patient 4 or a caregiver of the patient 4. For example, such instructions for medical intervention may include an instruction to change a drug dosage, timing, or selection, to schedule a visit with the clinician, or to seek medical attention. In further examples, server 14 may generate an alert to patient 4 based on a status of a medical condition of patient 4, which may enable patient 4 to proactively to seek medical attention prior to receiving instructions for medical intervention. In this manner, patient 4 may be empowered to take action, as needed, to address his or her medical status, which may help improve clinical outcomes for patient 4.

In the example illustrated in FIG. 3, external device 12 includes processor 122, storage device 124, and input/output circuitry 126; and server 14 includes a processor 142, a storage device 144, and input/output circuitry 146. Processors 122 and 142 may include one or more processors that are configured to implement functionality and/or process instructions for execution within external device 12 or server 14, respectively. For example, processor 122 may be capable of processing instructions stored in storage device 124; and processor 142 may be capable of processing instructions stored in storage device 144. Input/output circuitry 126 and 146 may include any suitable hardware, firmware, software, or any combination thereof for communicating with another device. In some examples, a description of a processor 122 or 142 outputting a signal, such as a diagnosis or alert, may include the processor causing its respective input/output circuitry to output the signal. Processors 122, 142 may include, for example, microprocessors, DSPs, ASICs, FPGAs, GPUs, or equivalent discrete or integrated logic circuitry, or a combination of any of the foregoing devices or circuitry. Accordingly, processors 122 and 142 may include any suitable structure, whether in hardware, software, firmware, or any combination thereof, to perform the functions ascribed herein to the respective processor.

Storage device 124, 144 may include a computer-readable storage medium or computer-readable storage device. In some examples, storage device 122 or 124 includes one or more of a short-term memory or a long-term memory. Storage device 124, 144 may include, for example, RAM, DRAM, SRAM, magnetic discs, optical discs, flash memories, or forms of EPROM or EEPROM. In some examples storage device 124 is used to store data indicative of instructions for execution by processor 122; and storage device 144 is used to store data indicative of instructions for execution by processor 142.

Although the techniques for triggering an ultrasound measurement based on a heart rhythm condition classification are described herein primarily (e.g., with respect to FIGS. 1-4) as being performed by processor 206 of cardiac monitor 10, such techniques may be performed, in whole or part, by a processor of any one or more devices of the system of FIG. 3, such as processor 122 of external device 12 and/or processor 142 of server 14.

One or more of processors 206, 122, or 142 may apply sensor data or feature vectors derived from sensor data, e.g., ECG or other cardiac activity data, to one or more models, e.g., machine learning models, to determine the occurrence or predict the occurrence, e.g., within a number of seconds or milliseconds, of AF or another heart rhythm condition/event. Based on the determination or prediction, the processor(s) may trigger capture of ultrasound images before, during, and/or after the heart rhythm condition. In some examples, the one or more models may implement regression, artificial intelligence, deep learning, and/or statistical processes to predict a heart rhythm condition/event.

Example machine learning techniques that may be employed to generate such models can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

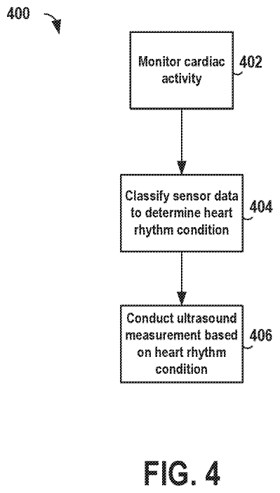

FIG. 4 is a flow chart illustrating an exemplary process 400 for using a cardiac monitor (e.g., cardiac monitor 10) in accordance with some aspects of this disclosure. As indicated by process 400, processor 206 may monitor cardiac activity (402) using a cardiac sensor 204. In various examples, the processor 206 may periodically, intermittently, or continuously use the cardiac sensor 204, wherein the cardiac sensor 204 is one of an EGM sensor or a PPG sensor configured for low-power operation compared to the ultrasound array 202.

Processor 206 may store information relating to the sensed cardiac activity in storage device 12, and/or may output information relating to the sensed cardiac activity using input/output circuitry 210. In some examples, processor 206 may analyze information relating to the sensed cardiac activity and may classify the sensor data to determine a heart rhythm condition (404). For example, processor 206 may employ a heart rhythm classification algorithm to recognize different arrhythmias such as AF based on the sensed cardiac activity. Based on the determined heart rhythm condition, the processor 206 may conduct an ultrasound measurement (406). For example, when the processor 206 detects an arrhythmia condition such as AF, the processor 206 may activate the ultrasound array 202 to measure one or more parameters of the patient's heart, including but not limited to structure, stroke volume, left ventricular ejection fraction, and other measurements. The processor 206 may store the ultrasound measurement information in storage device 212, analyze the ultrasound measurement information, and/or transmit the ultrasound measurement information using input/output circuitry 210. After the measurement of the one or more parameters associated with the detected event, the processor may then take steps to reduce the power consumption by ceasing utilization of the ultrasound array 202 by, for example, ceasing processing of data provided by the ultrasound array 202 to produce the measurements or by deactivating ultrasound array 202 entirely until detection of the next event (e.g., by a subsequent execution of step 404 in respond to cardiac activity captured at a later time).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an implantable medical device (IMD), an external programmer, a combination of an IMD and external programmer, an integrated circuit (IC) or a set of ICs, and/or discrete electrical circuitry, residing in an IMD and/or external programmer.

Various examples have been described. These and other examples are within the scope of the following claims.

Example 1. An apparatus comprising: a processor; a cardiac sensor; and an ultrasound sensor, wherein the processor is configured to: detect an onset of an arrhythmia condition using the cardiac sensor; and trigger an ultrasound measurement using the ultrasound sensor based on detection of the onset of the arrhythmia condition using the cardiac sensor.

Example 2. The apparatus of Example 1, wherein the cardiac sensor comprises one of a cardiac electrogram (EGM) sensor or a photoplethysmogram (PPG) sensor.

Example 3. The apparatus of Example 1 or 2, wherein the ultrasound measurement comprises one of a blood flow measurement or a cardiac image.

Example 4. The apparatus of any one or more of Examples 1 to 3, wherein the apparatus comprises a wearable device or a patch.

Example 5. The apparatus of any one or more of Examples 1 to 4, wherein the arrhythmia condition comprises atrial fibrillation.

1. An apparatus comprising:
a processor;
a cardiac sensor; and
an ultrasound sensor,
wherein the processor is configured to:
detect an onset of an arrhythmia condition using the cardiac sensor; and
trigger an ultrasound measurement using the ultrasound sensor based on detection of the onset of the arrhythmia condition using the cardiac sensor.

2. The apparatus of claim 1, wherein the cardiac sensor comprises one of a cardiac electrogram (EGM) sensor or a photoplethysmogram (PPG) sensor.

3. The apparatus of claim 1, wherein the ultrasound measurement comprises one of a blood flow measurement or a cardiac image.

4. The apparatus of claim 1, wherein the apparatus comprises a wearable device or a patch.

5. The apparatus of claim 1, wherein the arrhythmia condition comprises atrial fibrillation.

6. The apparatus of claim 1, further comprising a battery, wherein conducting the ultrasound measurement increases power consumption on the battery, and
wherein, subsequent to conducting the ultrasound measurement, the processor is configured to cease using the ultrasound sensor, whereby the power consumption on the battery is decreased.

7. A medical device system comprising:
an implantable medical device comprising:
a plurality of electrodes to configured detect a cardiac electrical signal of a patient,
communication circuitry configured to communicate with at least one other device; and
processing circuitry configured to:
identify a cardiac episode in the cardiac electrical signal of the patient, and
transmit an instruction for an external device to begin monitoring the cardiac episode via a sensor of the external device.

8. The medical device system of claim 7, wherein the implantable medical device comprises the processing circuitry.

9. The medical device system of claim 8, wherein, in transmitting the instruction, the processing circuitry is configured to transmit the instruction via the communication circuitry directly to the external device.

10. The medical device system of claim 7, wherein the cardiac episode is an arrythmia episode.

11. The medical device system of claim 10, wherein the arrythmia episode is an atrial fibrillation episode.

12. The medical device system of claim 7, wherein the sensor comprises an ultrasound sensor.

13. A sensor patch comprising:
a sensor device;
a power supply; and
a processor configured to:
receive an indication that a cardiac sensor has detected a cardiac event in a patient,
utilize the sensor device to produce a measurement of the cardiac event, whereby a power draw on the power supply is increased, and
cease utilization of the sensor device after the measurement of the cardiac event is produced, whereby a power draw on the power supply is decreased.

14. The sensor patch of claim 13, further comprising input/output circuitry, wherein the cardiac sensor is external to the sensor patch and the indication is received via the input/output circuitry.

15. The sensor patch of claim 13, wherein the sensor device comprises an ultrasound array.
